(19) United States
(12) Patent Application Publication
(10) Pub. No.: US 2026/0077127 A1
(43) Pub. Date: Mar. 19, 2026

(54) NOVEL ENHANCED PATCH PUMPS AND BIHORMONAL GLUCAGON PATCH CONTROL SYSTEMS

(71) Applicant: Beta Bionics, Inc., Irvine, CA (US)

(72) Inventors: Michael J. Rosinko, Peoria, AZ (US); Mike Mensinger, San Diego, CA (US); David Lim, Irvine, CA (US)

(73) Assignee: Beta Bionics, Inc., Irvine, CA (US)

(21) Appl. No.: 19/330,952

(22) Filed: Sep. 17, 2025

Related U.S. Application Data
(60) Provisional application No. 63/695,449, filed on Sep. 17, 2024.

(51) Int. Cl. A61M 5/168; A61K 9/00; A61K 9/70; A61K 38/26; A61K 38/28; A61M 5/14; A61M 5/142; A61M 5/158; A61M 5/172; G16H 40/67

(52) U.S. Cl. CPC A61M 5/16831; A61K 9/0009; A61K 9/703; A61K 38/26; A61K 38/28; A61M 5/1407; A61M 5/14248; A61M 5/1582; A61M 5/1723; G16H 40/67; A61M 2005/14268; A61M 2005/16863; A61M 2202/07; A61M 2205/18; A61M 2205/3569; A61M 2205/3584; A61M 2205/502; A61M 2205/8206; A61M 2230/201

(57) ABSTRACT

The new and future systems for low cognitive load disposable and durable medicament pumping systems now include at least one of pump embodiments for pairing of an APP in half-of a dozen embodiments respecting all known and to be installed features of the Beta Bionics (Irvine, CA patent estate and inchoate matters).

### CROSS-REFERENCE TO RELATED APPLICATIONS

This document claims the benefit of priority to US Provisional Application Ser. No. 63/695,449, filed Sep. 17, 2024, which is hereby incorporated by reference in its entirety.

### BACKGROUND OF THE ART

User preference and delivery of medicaments drive the optimization of many technologies involved in treating diabetes with infusion pumps.

Often slowing down the acceptance of better systems are the impediments overcome by the prior failed attempts. For infusion pumps the evolution has proceeded in reverse. First to patient were SYSTEMS FOCUSED ON HIGH LEVELS OF COGNITVE ENGAGEMENT—now understood to be a detriment or not needed to achieve best blood sugar results.

A new paradigm, it is respectfully proposed, takes the opposite approach. Beta Bionics have discovered that closed-loop systems for treating diabetes are manifested in patch pumps both in combination with extant BETA BIONICS systems (as referenced above) and in combination with third party insulin delivery methods. In essence, there a several principled approaches comprising combinations of novel drive train concepts with glucagon pumps which respond to glucose level control signals, inter alia.

For example, the Beta Bionics Bihormonal system using the iLET App for the Bionic Pancreas Patch Pump, and standalone systems working as accessories to third party insulin delivery systems are disclosed here and claimed below.

The principled basis for lower cognitive load for users and modular systems which require little or no user inputs have become an industry standard. For example, the iLET BIONIC PANCREAS with only the body weight of a user to prime said device delivers insulin as accurately as an system on the market with no keystrokes required.

### OBJECTS AND SUMMARY OF THE INVENTIONS

Briefly stated, any modular system aspect claimed and disclosed above in the list of pending patents is incorporated expressly by reference, and may be implemented, in whole or in part as the instant inventions or patch pumps, being the equivalent of any said patents and disclosures from the iLET brand of BIONIC PANCREAS system to any Bihormonal disclosures, including all needed software and algorithm interfacing to manage blood sugar control in identity with the other invention, combinations and subcombinations thereof.

According to embodiments of the instant disclosures, there are provided Infusion Schemata each operatively Linked and novelly configured according to the present invention with at least one of those systems re-designed to impart maximum user control with minimum input. Offered for consideration are at least about the following of which the instant system are comprised of—alone or in combination.
Dual cannula (“snakebite”)
Coaxial cannula
Top/bottom cannula
Micro-needle arrays.

Bihormonal Glucagon patch control schemas and implementations in several different forms, namely:
As part of the Beta Bionics Bihormonal system.
As part of a standalone system that would be an accessory to 3rd party insulin delivery systems.
Drivetrain concepts.
Single motor to drive two medicaments, one device (clutch, ratchet).
Nitinol wire approach to.

### BRIEF DESCRIPTION OF THE DRAWINGS

The systems, methods, and devices of this disclosure each have more than several innovative aspects, no single one of which is solely responsible for all of the desirable attributes disclosed herein. Details of one or more implementations of the subject matter described in this specification are set forth in the accompanying drawings and the description below, which are merely illustrative and not limiting, often omitting state of the art details for clarity of description, as known to those having a modicum of skill in the art.

FIG. 1 shows a cartooned schematic of an embodiment of a patch pump functioning as part of a patch pump system or kit for at least one of iLet Bionic Pancreas Patch Pump Bihormonal Glucagon Patch Pump and component of a third party insulin delivery system driven by the BETA BIONICS software and logic structures as listed in patents incorporated expressly by reference herein infra, including each proprietary elements of said data delivered by patch pumps,, according to the present inventions.

FIG. 2 shows a cartooned schematic of an example blood glucose control system embodied in present inventions.

FIG. 3 shows a cartooned schematic of an example blood glucose control system embodied in present inventions.

FIG. 4 shows a cartooned schematic of an example blood glucose control system embodied in present inventions.

FIG. 5 shows a cartooned schematic of an example blood glucose control system embodied in present inventions.

FIG. 6 shows a cartooned schematic of an example blood glucose control system embodied in present inventions.

FIG. 7 shows a cartooned schematic of an example blood glucose control system

FIG. 8 shows a cartooned schematic of an example blood glucose control system embodied in present inventions.

FIG. 9 shows a cartooned schematic of an example blood glucose control system embodied in present inventions.

FIG. 10 illustrates a partial plan view of an example ambulatory medical device being a novel enhanced Patch Pump system (PPs).

FIG. 11 illustrates a dorsal view showing sections removable for a combination of drable and disposable assembly view of the ambulatory medical device shown manifested in a novel enhanced patch pump configuration.

### DETAILED DESCRIPTIONS OF PREFERRED EMBODIMENTS

Infusion sets have been known to deliver medicaments for years. However, the challenges of rapid and concise developments have not been met. Early patents from U.S. Pat. Nos. 7,407,491 to 7,520,867 underscore the paucity of solutions to address the needs for steel cannula for 3-day sets and 7 day-sets either or in combination with soft cannula.

It is respectfully proposed that the novel configurations of our systems drive unique approaches based on providing novel enhanced system comprised of, in combination; a semi-reusable pump controlled by using an App operatively linked to an algorithm set and GUIs selectively for commanding interfacing with at least the pump, and standalone systems to simplify insulin delivery with minimal input and thus low to zero cognitive load imparted to the user.

Likewise taught are novel enhanced systems being further comprised of, in combination; a semi-reusable pump controlled by using an App operatively linked to an algorithm set and GUIs selectively for commanding interfacing with at least the pump, and standalone systems to simplify insulin delivery with minimal input and thus low to zero cognitive load from the user by prioritizing automation, seamless transitions and quick updates, having at least a twelve hour grace period with a standard 3-day work time.

The iLET Bionic Pancreas Patch Pump, and standalone systems working as accessories to third party insulin delivery systems are disclosed which leverage the drivetrain embodiments reduced to practice for these systems comprised of at least one of single motor to drive two medicaments, one device; Nitinol wire embodiments to drive two channels and multi-medicament systems; and chimeric versions all having simplified insulin delivery workflows and minimum user inputs and keystrokes.

Also taught is an infusion set disposable kit, comprising: infusion devices configured to link pumps and patients comprised of at least one of dual cannula (“snakebite”); coaxial cannula; top/bottom cannula and novel enhanced microneedle arrays.

The present inventors have discovered and herein disclose novelties driven by a new paradigm whereby patch pumps embody al of the features embodied in the software and logic structures emplaced within and transmitted to extant hardware developed for adaptive learning, with simplified and generified access to third party systems.

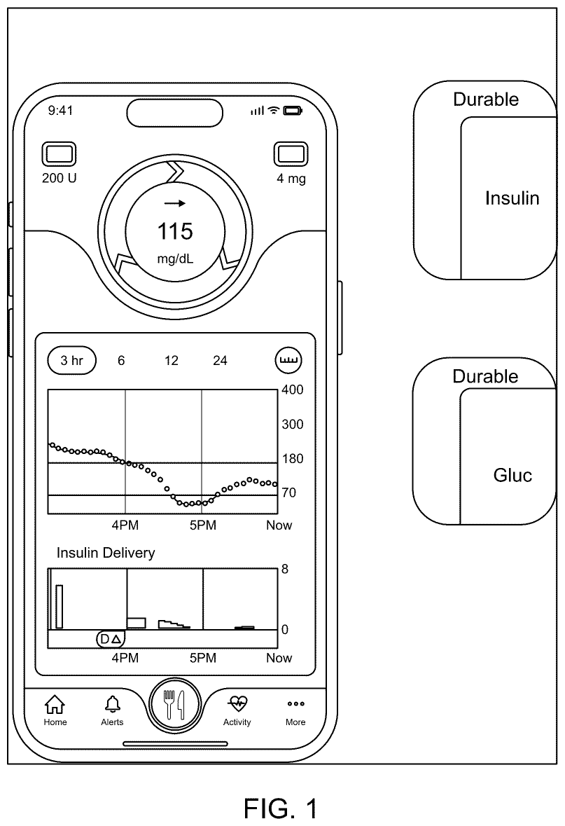

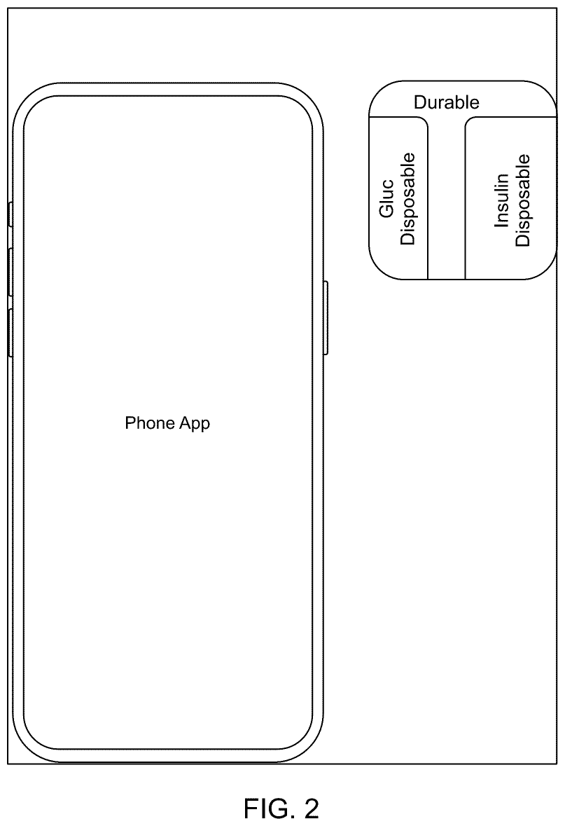

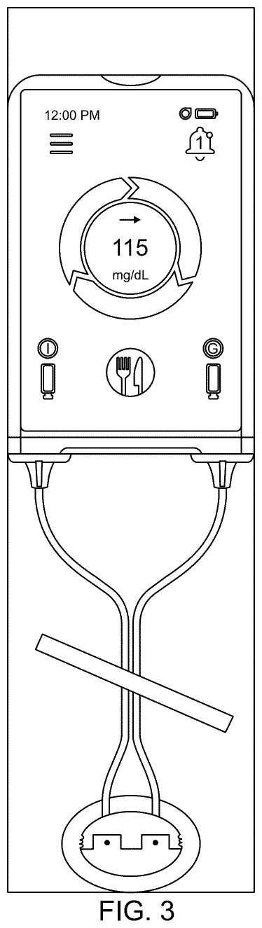

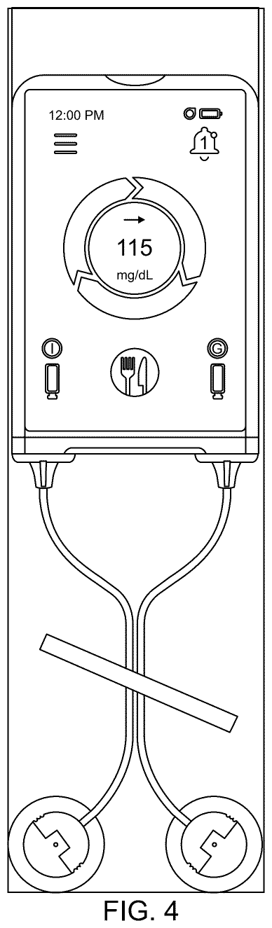

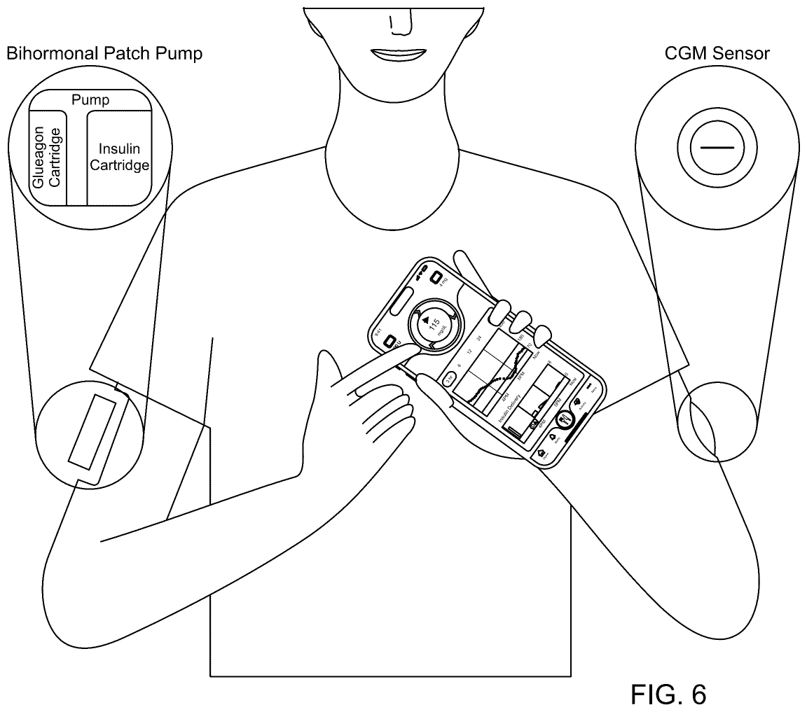

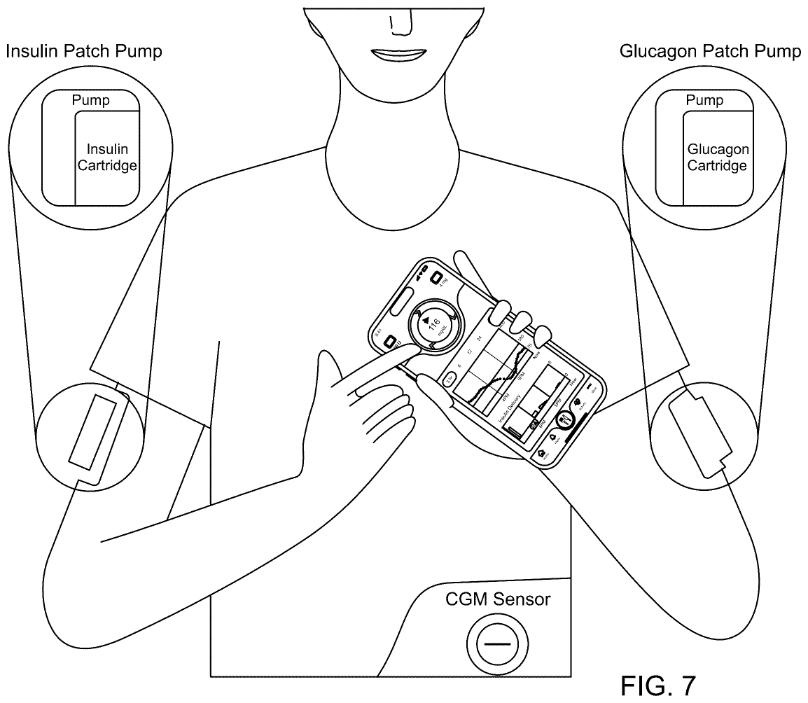

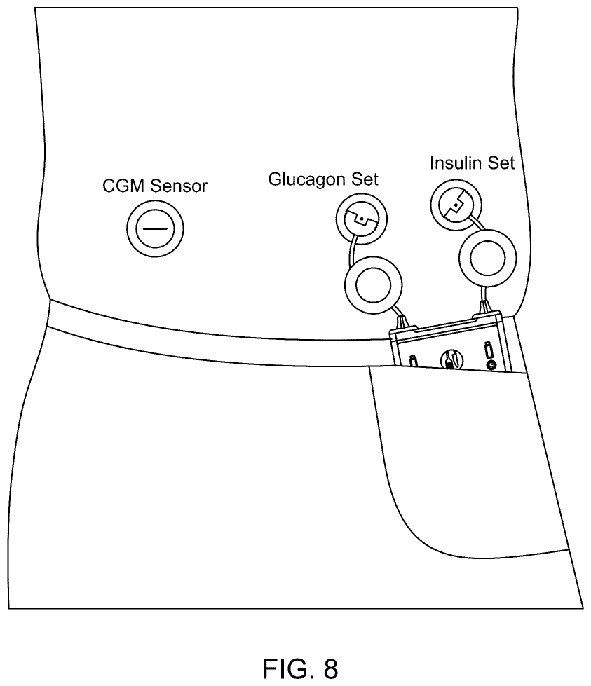

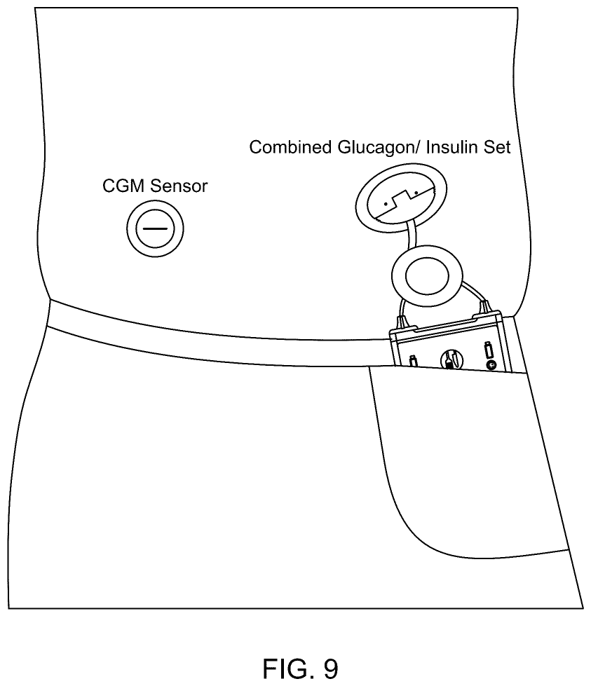

FIGS. 1 through 9 are integrated herein as previously disclosed and further explained in co-pending USSN at the USPTO, along with all of the table bound and listed patent and patent applications of Applicant from provisional patent applications associated herewith. In short, according to the instant teachings systems were patented from at least as early as December of 2020 for autonomous systems functioning based solely upon a priming dose associated with user's bodyweight with a learning algorithm on a patch.

Patch Pumps' transmission of adapted parameters is controlled by various triggers SEE for example BETA BIONICS' US Letters Patents Numbered U.S. Pat. Nos. 11,594,313; 11,688,501 and 11,610,661. User interactions keep patches up to date and infusion algorithms constantly improving. FIGS. 1-3 depicting myriad options such as 2 patches 2 cannulae to Boolean aggregates therefrom based in part on a first option being 2 sites, 2 cannulae and steel or Teflon/PTFE.

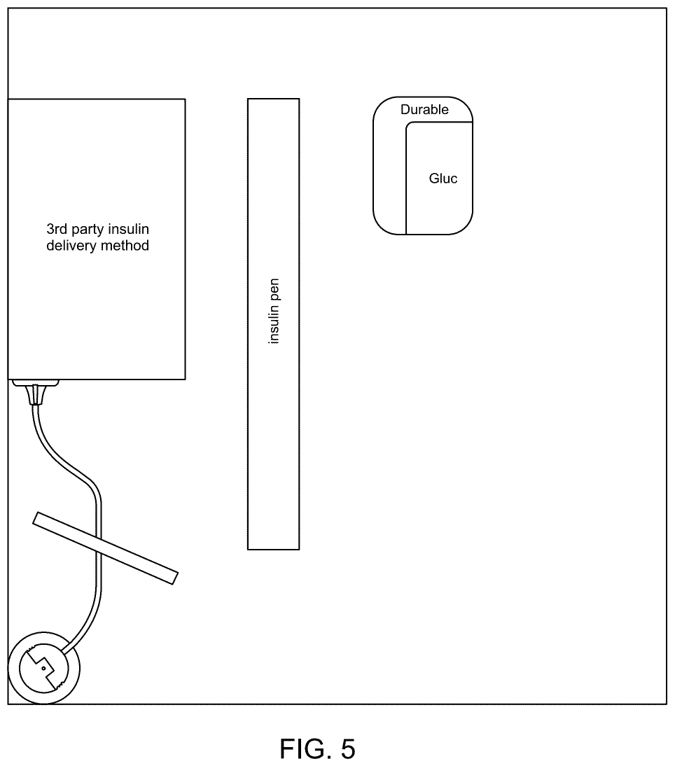

FIG. 5 through FIG. 9 highlight and underscore aspects of the instant teachings as applied to a novelty-based method for servicing third party insulin delivery methods. Artisans are well aware that each of the prior combinations may be used to address this approach, as explained and claimed below.

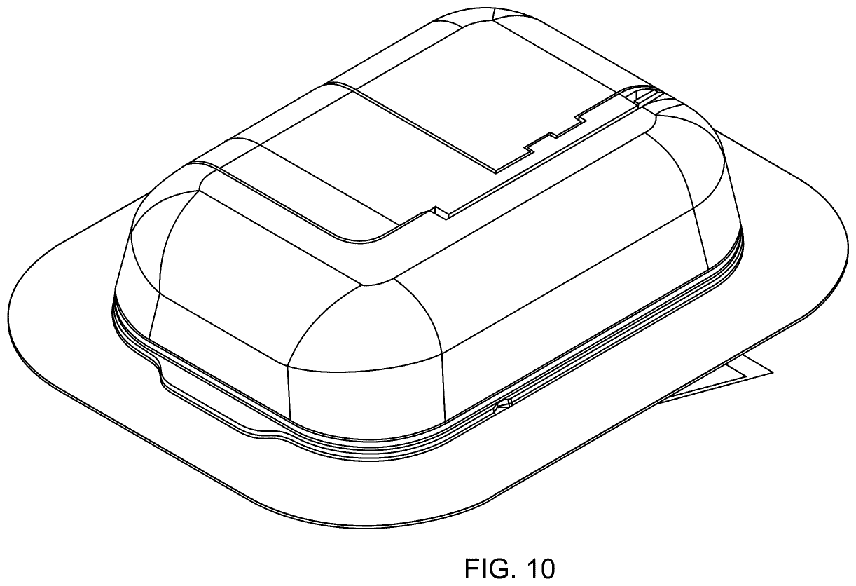

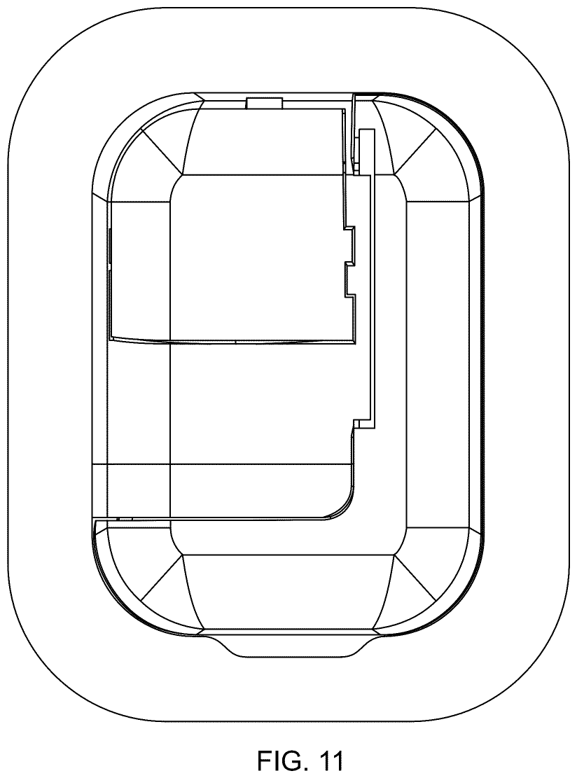

Referring now to FIG. 10 and FIG. 11 Patch Pump systems (PPs) are defined herein as at least one of the iLET BIONIC PANCREAS Patch Pump system, the iLET BIONIC PANCREAS Patch Pump Bihormonal system and the iLET BIONIC PANCREAS Patch Pump system, a tubeless ACEpump, there are shown a bottom adhesive section for housing at least one of a steel cannulae, Teflon (PTFE) cannulae, micro-needle arrays to provide ingress to the patient/user. As is known, these new infusion pumps can be small, light-weight and easy to use. Such Patch pumps stand as improvements in certain clinical settings, but have enumerated strong needs to improve fluid driving mechanisms from reservoirs through transcutaneous access tools the present inventors hereby offer for consideration one set of such systems, as expressly labeled as desiderata by industry, including over the INSULET OMNIPOD brand of device (see U.S. Pat. No. 10,420,883 Column 2, line 2).

### Examples One and Two the Patch Pump Inventions

Beta Bionics have discovered that closed-loop systems for treating diabetes are manifested in patch pumps both in combination with extant BETA BIONICS systems (as referenced above) and in combination with third party insulin delivery methods.

In essence, there a several principled approaches comprising combinations of novel drive train concepts with glucagon pumps which respond to glucose level control signals, inter alia.

For example, the Beta Bionics Bihormonal system using the iLET App for the Bionic Pancreas Patch Pump, and standalone systems working as accessories to third party insulin delivery systems are disclosed here and claimed below.

The iLET Bionic Pancreas Patch Pump, and standalone systems working as accessories to third party insulin delivery systems are disclosed and claimed herein. FIGS. 5-8 describe these embodiments to those skilled in the art and constitute progress in science and the useful art.

Novelties of the iLET App for the Bionic Pancreas Patch Pump, and standalone systems working as accessories to third party insulin delivery systems are disclosed which leverage the drivetrain embodiments reduced to practice for these systems comprised of single motor to drive two medicaments, one device (clutch, ratchet); Nitinol wire embodiments to drive two channels and multi-medicament systems; and chimeric disclosures including those dis closed in co-pending 63/627,278 of BETA BIONICS expressly incorporated herein by reference as if full set forth herein.

With respect to the iLET App for the Bionic Pancreas Patch Pump and the patch pump itself, plethoric infusion approaches have been configured comprised of dual cannula (“snakebite”); coaxial cannula; top/bottom cannula and novel enhanced microneedle arrays Likewise, features such as Silent Mode which allows the user to temporarily silence all audio tones and vibration alerts for sensor glucose alerts except Urgent Low Glucose (CGM glucose<54 mg/dl) and insulin delivery stoppages (all alarms related to when dosing has stopped, such as occlusions, empty cartridges, end of BG Run mode, and iLet loss of power). This may be useful in situations where users don't want to disturb others, such as at major life events (e.g., weddings and funerals), business meetings, movies, etc.

### Design

While in Silent Mode, the iLet will still display any active alerts in the GUI, and also record the time and glucose values during any alerts that occur. A record of all alarms will continue to be visible on the History screen. For the exceptions from Silent Mode, Urgent Low Glucose and insulin delivery stoppages, those alerts will be unaffected by Silent Mode and work as they currently do.

### UX/UI Concept

The iLet ACE Pump notifies users with audio tones and/or vibration alerts when attention is required. A bell-shaped Notification icon appears in the status bar, next to the battery icon. A visible message appears in the Notifications icon with the number of currently active alerts.

Users can enable Silent Mode from the Menu screen. Users can set the duration of silent mode for up to 8 hours in 15-minute increments. When Silent Mode is enabled, the Home Screen will have an indication that Silent Mode is on. Users will have the option to cancel Silent Mode at any time to switch back into previous alert notification volume settings or, the iLet will automatically switch back to its previous settings once the set time is up.
Silent Mode:

### Example Three

### Design

The Therapy Restore feature was designed to be used during activation of a replacement iLet ACE Pump and may be used within 30 days of the last available data upload to the Beta Bionics Cloud. The feature will only be available to users of the iLet App. When users pair their existing iLet App Account with their replacement iLet ACE Pump, they will be presented with the option to select Therapy Restore and restore from their last known point (date) or start over. They will also have the option to start the iLet without the use of Therapy Restore. If it has been more than 30 days since their last available data upload, users will not be presented with the Therapy Restore option.

Initializing insulin delivery will be the same for either the Therapy Restore option or starting over from factory settings. Users will be walked through standard device set up—including confirming their bodyweight. Users will be required to set up their CGM before setting up the replacement iLet ACE Pump with enough time to allow the CGM sensor to warm up. Just like with a new pump, the pump cannot be initialized until the CGM sensor is warmed up and displaying a glucose value.

Selecting Therapy Restore will restore the user's CGM Target setting, Secondary CGM Target setting (if utilized), bodyweight, and meal announcement adaptation (Usual for me Breakfast, Lunch, and Dinner). Users will be asked to confirm their bodyweight prior to initialization. Basal rate will not be restored, so basal insulin dosing will begin based on their body weight.

### Discussion

The Therapy Restore feature was designed in response to requests from users and HCPs asking for a feature that would allow return to steady state glucose control on the iLet as quickly as possible if the iLet had to be replaced for any reason. Although adaptation of the Basal and Correction algorithms occurs quickly, meal dose adaption may take up to one week because of the limited number of meals announced and therefore the limited number of opportunities to adapt the meal doses. Therapy Restore will initialize the iLet with the previously adapted meal doses to allow users to short-cut the part of adaptation that takes the longest.

In contrast to meal doses, adaptation of the basal and correction algorithms occur very rapidly. In the Bionic Pancreas Pivotal Trial participants randomized to the iLet arm reached their new time in range by 48 hours after initialization, on average. Most of the increase in time in range occurred in the first 24 hours. This is due to the speed of adaptation of the basal and correction algorithms. Therefore, it is unnecessary to restore the nominal basal profile or the aggressiveness parameter for the Correction algorithm to return quickly to the users' steady state glucose control. It is more conservative to return to the weight-based initialization settings for these algorithms, which are low for most users and therefore conservative, and that were validated in the Bionic Pancreas Pivotal Trial as being safe.

It is no less safe to restart the iLet using previously adapted meal doses than to start the iLet from factory settings and require the meal doses to adapt. We have chosen to limit the use of Therapy Restore to individuals with adapted meal doses that have been updated no longer than 30 days prior to starting the new iLet. This should ensure that there has been no significant changes in the insulin needs of the user since the meal doses were updated. Thirty days is one-third of the shortest typical interval between endocrinologist visits, which is the shortest interval between updates to the carbohydrate-to-insulin ratio and carbohydrate counting. In practice, the carbohydrate-to-insulin ratio is typically updated much less frequently than every three months, so a 30-day period to allow use of previously adapted meal doses is very conservative. In addition, the iLet gives 75% of the insulin predicted to be needed for a meal, providing a margin of safety.

If the user or the HCP is aware of significant change in the meal insulin need during the interval between the time the last update of iLet data to the cloud and the start of the new iLet, there is always the option to start the iLet using factory settings.

### Example Four

### iLet Dosing Decision Software: iLet App Control

### Design

The iLet App supports the use of either the Bionic Pancreas Patch Pump Syster or the iLet Bionic Pancreas System or DUO /ihormonal systems. In addition to its current functions (uploading user data and updating the iLet ACE Pump software), the proposed iLet App will add the same capabilities and function as the current iLet ACE Pump Graphic User Interface (GUI).

### Ilet App Features

Pair a CGM (critical task)
Enter Body Weight & Go Bionic (critical task)
View Home Screen
Adjust Volume (critical task)
View Graph and Therapy Summary (critical task)
Update Body Weight (critical task)
Bolus Insulin for Carbohydrates (Meal Announcement) (critical task)
Update CGM Target (critical task)
Update Secondary CGM Target (critical task)
Sensor goes offline, enter BG to continue dosing (BG-Run mode) (critical task)
Access History
Alarm/Alert Comprehension (critical task)
Pause Insulin after Disconnecting from Infusion Set
Turn on Critical Alert Setting in App (critical task)
Enable Exercise Mode (critical task)
Enable Silent Mode (critical task)
Pair with a pump

1. The novel enhanced system comprised of, in combination; a semi-reusable pump controlled by using an App operatively linked to an algorithm set and GUIs selectively for commanding interfacing with at least the pump, and standalone systems to simplify insulin delivery with minimal input and thus low to zero cognitive load imparted to the user.

2. Novel enhanced system of claim 1 being further comprised of, in combination; a semi-reusable pump controlled by using an App operatively linked to an algorithm set and GUIs selectively for commanding interfacing with at least the pump, and standalone systems to simplify insulin delivery with minimal input and thus low to zero cognitive load from the user by prioritizing automation, seamless transitions and quick updates.

3. Novel enhanced system of claim 2, having at least a twelve hour grace period with a standard 3-day work time.

4. Novel enhanced system of claim 2, for an improved grace period and 7-day set.

5. Novel enhanced system of claim 2, for longer resident sets.

6. The iLET Bionic Pancreas Patch Pump, and standalone systems working as accessories to third party insulin delivery systems are disclosed which leverage the drivetrain embodiments reduced to practice for these systems comprised of at least one of single motor to drive two medicaments, one device; Nitinol wire embodiments to drive two channels and multimedicaments systems; and chimeric versions all having simplified insulin delivery workflows and minimum user inputs and keystrokes.

7. An infusion set disposable kit, comprising: infusion devices configured to link pumps and patients comprised of at least one of dual cannula (“snakebite”); coaxial cannula; top/ bottom cannula and novel enhanced microneedle arrays.

8. The infusion set disposable kit of claim 7, in at least one of the following configurations:
2 patches, 2 cannulas;
1 patch, 2 cannulas;
1 site, 2 cannulas, steel only;
2 sites, 2 cannulas steel or Teflon.

9. The infusion set disposable kit of claim 7, disposed in at least one of the following configurations:
Standalone glucagon pump & 3rd party insulin delivery;
2 patches, 2 cannulas;
1 patch, 2 cannulas;
1 site, 2 cannulas, steel only; 2 sites, 2 cannulas steel or Teflon.

10. The iLET Bionic Pancreas Patch Pump of claim 6, further comprising at least a glucagon pump responding to glucose level signals and control from sensors integrated with manual use of insulin and the like medicaments.

11. The patch pump of claim 6, driven by response to glucose level control signals for insulin only.

12. The patch pump of claim 6, driven by response to glucose level control signals for glucagon or glucagon plus meaning enhanced DMSO—enriched moieties proprietary formulations used by license.

13. The novel enhanced system of claim 2, driven by response to glucose level control signals for any medicament combination or sole agent.

14. Novel enhanced patch pump driven by response to glucose level control signals for insulin only being AUTO-MAGICAL or it just works when a user walks away from app it switches automatically to the pump.

15. Novel enhanced patch pump driven by response to glucose level control signals for insulin only, of claim 14, and insulin plus glucagon and other medicaments further comprising the condition state of disconnection all alerts being reversion to user set volume level.

16. Novel enhanced patch pump driven by response to glucose level control signals for insulin only of claim 15, alarm annunciation and prioritization being identical to the current iLET Bionic Pancreas System.

17. Novel enhanced patch pump of claims above driven by response to glucose level control signals for insulin only of claim 16, alarm annunciation and prioritization if iLET device does not receive annunciation from the phone then the pump would need to annunciate; wherein the device is paired with at least one of an App or another patch and pump and via telecommunications and software linked to user designated resources.

18. Novel enhanced patch pump driven by response to glucose level control signals for insulin only of claim 17, having specialized alarm annunciation for occlusions—for example—and prioritization revisions with two options, user selected:
Some alerts where it clears on both displays—others where it does not.

19. Novel enhanced system of claim 1, driven by response to glucose level control signals for insulin only combined with software and algorithms to clear alerts and generate geo-location hierarchy lists such as in the case of progressive caregiver lists, or having them configured for nudging users toward, at least one behavior from the group comprising of:
Bolus Meal Announcements;
Low battery with patch issues;
CGM interface/power/control issues;
Therapy restore via Algorithm State Restore; and
And any patch pump related third party service monitoring and proximity network.

20. Novel enhanced system of claim 1, driven by response to glucose level control signals for insulin and glucagon or glucagon plus (glucagon plus meaning enhanced DMSO—enriched moieties proprietary formulations used by license)combined with software and algorithms to clear alerts and generate geo-location hierarchy lists such as in the case of progressive caregiver lists, or having them configured for nudging users toward, at least one behavior from the group comprising of:
Bolus Meal Announcements;
Low battery with patch issues;
CGM interface/power/control issues;
Therapy restore via Algorithm State Restore; and
And any patch pump related third party service monitoring and proximity network.
